(19) United States
(12) Patent Application Publication
(10) Pub. No.: US 2024/0198131 A1
(43) Pub. Date: Jun. 20, 2024

(54) UV A LIGHT EXPOSURE INCREASES MITOCHONDRIAL ANTI-VIRAL PROTEIN EXPRESSION IN TRACHEAL CELLS VIA CELL-TO-CELL COMMUNICATION AND USES THEREOF

(71) Applicant: Cedars-Sinai Medical Center, Los Angeles, CA (US)

(72) Inventors: Mark Pimentel, Los Angeles, CA (US); Ali Rezaie, Beverly Hills, CA (US); Gabriela Guimaraes Sousa Leite, Porter Ranch, CA (US); Ruchi Mathur, Los Angeles, CA (US); Gil Y. Melmed, Los Angeles, CA (US)

(73) Assignee: Cedars-Sinai Medical Center, Los Angeles, CA (US)

(21) Appl. No.: 18/288,146

(22) PCT Filed: May 5, 2022
(86) PCT No.: PCT/US2022/027915

§ 371 Date: Oct. 24, 2023

Related U.S. Application Data
(60) Provisional application No. 63/184,749, filed on May 5, 2021.

(51) Int. Cl. A61N 5/06; G01N 33/68

(52) U.S. Cl. CPC A61N 5/0624; G01N 33/6893; A61N 2005/0604; A61N 2005/0606; A61N 2005/0607; A61N 2005/0608; A61N 2005/061; A61N 2005/0611; A61N 2005/0661; G01N 2800/52

(57) ABSTRACT

Mitochondrial antiviral signaling (MA VS) protein mediates innate antiviral responses, and is an important component of the response to severe acute respiratory syndrome coronavirus-2 (SARS-COV-2). Herein methods are provided to increase expression of mitochondrial antiviral signaling (MAVS) protein in epithelial cells by use of a UVA therapy to expose the epithelial cells to the UVA therapy, or to contact a first set of epithelial cells with a second set of epithelial cells which have been exposed to the UVA therapy, or to contact a first set of epithelial cells with the cell lysates of the second set of epithelial cells that have been exposed to the UVA therapy.

### CROSS-REFERENCE TO RELATED APPLICATIONS

This application includes a claim of priority under 35 U.S.C. § 119(e) to U.S. provisional patent application No. 63/184,749, filed May 5, 2021, the entirety of which is hereby incorporated by reference.

### FIELD OF INVENTION

This invention relates to systems and methods for ultraviolet therapy to treat respiratory infectious diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The human body has many defenses against infection, the most well-known of which involve innate immune responses in which immune cells are recruited to sites of infection via cytokine signaling. However, intracellular responses to infection are also important, particularly in the defense against viruses. In the past decade, it has emerged that mitochondria can mediate the establishment and maintenance of innate and adaptive immune responses, including through the production of mitochondrial anti-viral (MAVS, or mitochondrial antiviral signaling) protein.

The MAVS protein is primarily localized to the outer membrane of the mitochondria, and transduces signals from RIG-I-like receptors (RLRs), which are cytoplasmic receptors that recognize viral RNA. Specifically, after recognition and binding of viral components, the RLRs retinoic acid-inducible gene I (RIG-I) and melanoma differentiation-associated gene 5 (MDA5) interact with MAVS, activating transcription factors that induce expression of proinflammatory factors and antiviral genes. However, some viruses have developed mechanisms to antagonize the activation of MAVS and evade this innate immune response. For example, the SARS-COV-2 transmembrane glycoprotein M is thought to antagonize MAVS, thus impairing MA VS-mediated innate antiviral responses.

### SUMMARY OF THE INVENTION

The following embodiments and aspects thereof are described and illustrated in conjunction with compositions and methods which are meant to be exemplary and illustrative, not limiting in scope.

Methods of increasing expression of mitochondrial antiviral signaling (MAVS) protein in epithelial cells are provided, including exposing epithelial cells to an effective amount of ultraviolet A (UVA) light, so as to increase expression of MAVS protein in the epithelial cells or in distant epithelial cells unexposed to the effective amount of UVA. Preferably the methods are performed in a subject in need thereof; and the methods, using UV exposure at least in the 335-350 nm, do not cause UV-induced DNA damage to the exposed cells, and do not require an administration of anesthesia to the subject before, during, or after the UVA light exposure. In various aspects, the increase in MAVS protein expression is compared to not having been exposed to the effective amount of the UVA or compared to a control. The control may be a reference value of the epithelial cells before exposure to the UVA, epithelial cells before contact with a pathogen, or population of epithelial cells not exposed to the amount of the UVA and not infected with a pathogen.

In some embodiments, the epithelial cells comprise tracheal epithelial cells, nasopharyngeal epithelial cells, ciliated epithelial cells. In some embodiments, the epithelial cells are one or more of mammalian nasal epithelial cells, mammalian oral epithelial cells, mammalian olfactory epithelial cells, mammalian trachea epithelial cells, mammalian pharyngeal epithelial cells, mammalian lung epithelial cells. In additional embodiments, the epithelial cells are urethral epithelial cells, bladder epithelial cells, vaginal epithelial cells, urogenital epithelial cells, gastrointestinal epithelial cells (e.g., rectal epithelial cells, or gastrointestinal epithelial cells other than rectal epithelial cells), outer ear epithelial cells, and/or middle ear epithelial cells.

In some embodiments of the methods, exposing tracheal epithelial cells of a subject to the UVA light, or irradiating the tracheal epithelium with the UVA light, increases the MAVS protein expression in the trachea of the subject.

In some embodiments of the methods, exposing nasal epithelial cells, olfactory epithelial cells, oral epithelial cells, or combinations thereof of a subject to the UVA light, or irradiating the nasal epithelium, olfactory epithelium, and/or oral mucosal epithelium of a subject with the UVA light, increases the MAVS protein expression in the nasal epithelium, olfactory epithelium, and/or oral mucosal epithelium of the subject, as well as in the subject's lung.

As additional examples of increasing the expression of MAVS protein in distant epithelial cells (including those unexposed to the UVA light), the methods in some embodiments may include that exposing urethral epithelial cells to the UVA light increases MAVS protein expression in epithelial cells in the subject's bladder; exposing vaginal epithelial cells to the UVA light increases MAVS protein expression in epithelial cells in the subject's uterus; exposing penile epithelial cells to the UVA light increases MAVS protein expression in epithelial cells in the subject's urethra or bladder; exposing rectal epithelial cells to the UVA light increases MAVS protein expression in epithelial cells in the subject's rectum or colon; exposing outer ear epithelial cells to the UVA light increases MAVS protein expression in epithelial cells in the subject's middle or inner ear; and/or exposing middle ear epithelial cells to the UVA light increases MAVS protein expression in epithelial cells in the subject's inner ear.

In some embodiments, a subject in need of or undergoing the UVA light exposure does not have a symptom or sign of a microbial infection, or has not been exposed to a microbial infection. In some embodiments, a subject in need of or undergoing the UVA light exposure exhibits a symptom or sign of a microbial infection for no more than 3 days, 5 days, 7 days, or 10 days. In additional embodiments, the methods further include selecting the subject who exhibits a symptom or sign of the microbial infections as the subject in need of the UVA light exposure, before exposing his/her epithelial cells to an effective amount of the UVA light.

Some embodiments provide that an effective amount of the UVA light increases the MAVS protein level, so as to reduce proliferation of a microbe that has infected the epithelial cells, or to pre-treat the epithelial cells before a microbial infection so that a microbial infection will have a lower proliferation rate, or even decreased amount, when infecting the epithelial cells. An effective amount of the UVA light administration may, in some embodiments, include one or more continuous exposures, or one or more pulse exposures.

Methods of assessing UVA treatment in a subject in need thereof are also provided, which include assaying a biological sample obtained from a subject having been exposed to UVA treatment for MAVS protein expression level, wherein a MAVS protein expression level higher than the subject's baseline level or higher than a control level indicates the treatment being effective. The biological sample, in various implementations, include epithelial cells.

Methods of administering UVA treatment in a subject in need thereof are further provided, which include assaying MAVS protein expression in a biological sample obtained from a subject having been exposed to UVA treatment, and continuing to administer UVA treatment to the subject if MAVS protein expression is lower than the subject's baseline level, compared to a control, or compared to a target level.

In some embodiments, a method of administering ultraviolet A (UVA) treatment in a subject in need thereof includes exposing epithelial cells to an effective amount of ultraviolet A (UVA) in a subject having a low MAVS protein expression as compared to a control, which is indicative of the subject needing the UVA treatment, wherein the exposure increase expression of MAVS protein in the epithelial cells or in distant epithelial cells unexposed to the effective amount of UVA. In other embodiments, a method of administering ultraviolet A (UVA) treatment in a subject in need thereof includes exposing epithelial cells to an effective amount of ultraviolet A (UVA) in a subject having a MAVS protein expression higher than the subject's baseline level, or compared to a control, which is indicative of the UVA treatment being effective.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A depicts a schematic showing the design of experiments in which 100% confluent monolayer plates of primary tracheal epithelial cells (HTEpC) were partially exposed to 2 mW/cm² NB-UVA for 20 minutes. NB-UVA was only applied to area 1. After UVA therapy, cells were collected from areas 4, 3, 2 and 1 in that order.

FIG. 1B depicts normalized MAVS levels in 30-40% confluent HTEpC exposed to 2 mW/cm² NB-UVA for 20 minutes, and in unexposed controls. Y-axis unit is AU (arbitrary units) normalized by Ponceau.

FIG. 1C depicts normalized MAVS levels in 100% confluent HTEpC area 1 exposed to 2 mW² mW/cm² NB-UVA for 20 minutes and in unexposed monolayer controls. Y-axis unit is AU (arbitrary units) normalized by Ponceau.

FIG. 1D depicts normalized MAVS levels in 30-40% confluent naïve HTEpC treated with supernatants from 30-40% confluent NB-UVA exposed HTEpC, and in controls incubated with supernatants from unexposed 30-40% confluent HTEpC.

FIG. 1E depicts western blot of proteins extracted from 30-40% confluent naïve HTEpC treated with supernatant from 30-40% confluent NB-UVA exposed HTEpC (lanes 1, 2 and 3), and from controls treated with supernatant from 30-40% confluent unexposed HTEpC (lanes 4, 5 and 6).

FIG. 1F depicts normalized MAVS levels in 30-40% confluent naïve HTEpC treated with lysates from 30-40% confluent NB-UVA exposed HTEpC, and in controls incubated with lysates from 30-40% confluent unexposed HTEpC.

FIG. 1G depicts western blot prepared directly from lysates of 30-40% confluent naïve HTEpC incubated with lysates from 30-40% confluent NB-UVA exposed cells (lanes 1 to 4) and from lysates of controls incubated with lysates from 30-40% confluent unexposed HTEpC (lanes 5 to 8).

FIG. 1H depicts normalized MAVS levels in 100% confluent HTEpC partially exposed to 2 mW/cm² NB-UVA for 20 minutes. Area 1 was directly exposed to NB-UVA, but areas 2, 3 and 4 were not exposed to NB-UVA.

FIG. 1I depicts western blot prepared from cell lysates of 100% confluent HTEpC from three experiments, exposed to NB-UVA (area 1-lanes 1, 5 and 9) and from lysates of confluent HTEpC not exposed to NB-UVA from the same culture plate (area 2-lanes 2, 6 and 10; area 3-lanes 3, 7 and 11; area 4-lanes 4, 8, and 12.

FIG. 1J depicts western blot of proteins extracted from 100% confluent HTEpC exposed to NB-UVA (lanes 1, 2 and 4), and 100% confluent HTEpC that were not exposed to NB-UVA (Lanes 5, 6 and 7). Lane 3 (exposed to NB-UVA) was discarded due to poor total protein magnification.

FIG. 2 depicts normalized MAVS levels in 30-40% confluent HTEPC cells exposed to 2 mW/cm² NB-UVA for 20 minutes (1, 2 and 3 times), in HTEpC cells exposed to 5 mW/cm² NB-UVA for 20 minutes (1 time), and in unexposed controls.

FIG. 3A depicts normalized MAVS levels in 100% confluent HTEpC cells exposed to 2 mW/cm² NB-UVA for 20 minutes. FIG. 3B shows western blot of proteins extracted from 100% confluent HTEpC cells exposed to NB-UVA (lanes 1 to 4) and 100% confluent HTEpC cells that were not exposed to NB-UVA (lanes 5 to 7).

FIG. 4A depicts normalized MAVS levels in HTEpC cells incubated with lysates from NB-UVA exposed HTEpC cells and in controls incubated with lysates from unexposed HTEpC cells. FIG. 4B shows western blot prepared directly from lysates of HTEpC cells incubated with lysates from NB-UVA-exposed cells (lanes 1 to 5) and from lysates of control HTEpC cells incubated with lysates from unexposed cells.

FIG. 5A depicts normalized MAVS levels in 100% confluent HTEpC cells partially exposed to 2 mW/cm² NB-UVA for 20 minutes. Area 1 was directly exposed to NB-UVA, but Areas 2, 3 and 4 were not exposed to NB-UVA. FIG. 5B shows western blot prepared from cell lysates of 100% confluent HTEpC cells exposed to NB-UVA (Area 1-lanes 1, 5 and 9) and from lysates of confluent HTEpC cells not exposed to NB-UVA from the same culture plate (Areas 2, 3 and 4-lanes 2, 3, 4, 6, 7, 8, and 10, 11 and 12, respectively).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, N Y 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein the phrase “distant” with reference to epithelial cells refers to epithelial cells that are directly connected (e.g., by gap junctions, tight junction or desmosomes) or indirectly connected to UVA exposed epithelial cells. Indirect connection in this context refers to cells that are connected cell to cell to cell which are eventually directly connected with UVA exposed epithelial cells.

In various embodiments, a distant epithelial cell is up to than 30 cm away from the periphery of UVA light-exposed/irradiated area or volume of epithelial cells. In various embodiments, a distant epithelial cell is up to than 20 cm away from the periphery of UVA light-exposed/irradiated area or volume of epithelial cells. In various embodiments, a distant epithelial cell is up to than 10 cm away from the periphery of UVA light-exposed/irradiated area or volume of epithelial cells. In various embodiments, a distant epithelial cell is up to than 5 cm away from the periphery of UVA light-exposed/irradiated area or volume of epithelial cells.

“Mitochondrial antiviral-signaling protein (MAVS),” also known as IPS1, KIAA1271, VISA, or CARDIF, (Protein Accession number Q7Z434), is a 540 amino acid protein that contains one caspase-recruitment domain (CARD) and several transmembrane domains, and localizes to the outer mitochondrial membrane. Without wishing to be bound by a particular theory, MAVS is believed to function downstream of proteins, such as retinoic acid-inducible gene (RIG-I), that detect double-stranded (ds) viral replication, and be required for proper immune response against ds viral infection.

As part of our work to explore the potential of ultraviolet light A (UVA) therapy in treating infections, we recently showed that application of UVA light, under specific conditions, to human ciliated tracheal epithelial cells infected with coronavirus-229E significantly improved cell viability and prevented virus-induced cell death, and that this was accompanied by decreases in the levels of CoV-229E spike (S) protein. Moreover, cells treated with UVA light exhibited significantly increased levels of MAVS protein, which indicated that UVA may active MAVS. Further, in a first human clinical trial of the safety of using endotracheally-delivered UVA light to treat mechanically ventilated subjects with coronavirus disease 2019 (COVID-19), subjects exhibited significant decreases in SARS-COV-2 viral loads in endotracheal aspirates by day 6 of therapy, indicating that UVA light could decrease SARS-CoV-2 infection. Moreover, there was also a decrease in viral loads in nasal swabs, despite the fact that only small portions of the trachea were exposed to UVA light.

In this study, we explored the effects of UVA light (such as narrow band, NB, UVA light) on MAVS expression in human ciliated tracheal epithelial cells in vitro. We also explored whether the effects of UVA light were limited to cells directly exposed to UVA, or were also seen in cells not directly exposed to UVA.

Generally, a narrow band (NB) UV-A (or UVA, or UV A) light is centered around 345 nm in wavelength and can include a range of ±1 nm, ±2 nm, ±3 nm, ±4 nm or ±5 nm. In some embodiments, a NB UV-A has a peak wavelength in a range from 343 nm to 345 nm. In some embodiments, a NB-UVA LED is used and it emits a peak wavelength in a range from 343 nm to 345 nm. In some embodiments, UV-A light is between 315 nm and 400 nm, or between 320 nm and 410 nm, or between 335 nm and 350 nm. In some embodiments, UV-A light peaks between 335 nm and 345 nm. In some examples, a light source is used which is an LED with a peak wavelength of 341 nm, 342 nm, 343 nm, 344 nm, 345 nm, 346 nm, 347 nm, 348 nm, and/or 349 nm. In some examples, the peak wavelength of an LED may have a +/−3 nm, 2 nm, or 1 nm error around them. In some embodiments, only UV-A light is exposed to the tracheal cells of a subject.

Various embodiments provide methods of increasing expression of mitochondrial antiviral signaling (MAVS) protein in epithelial cells in a subject in need thereof, which include: exposing epithelial cells to an effective amount of ultraviolet A (UVA), so as to increase expression of MAVS protein in the epithelial cells or in distant epithelial cells unexposed to the effective amount of UVA, wherein the increased expression of MAVS protein is compared to not having been exposed to the effective amount of the UVA or compared to a control.

In some implementations, a method is provided for increasing MAVS protein expression in epithelial cells in a subject in need thereof by exposing epithelial cells to an effective amount of UVA for increasing expression of MAVS protein in the epithelial cells.

In some implementations, a method is provided for increasing MAVS protein expression in epithelial cells in a subject in need thereof by exposing epithelial cells to an effective amount of UVA for increasing expression of MAVS protein in distant epithelial cells unexposed to the effective amount of UVA.

In other implementations, a method is provided for increasing MAVS protein expression in epithelial cells in a subject in need thereof by exposing epithelial cells to an effective amount of UVA, so as to increase expression of MAVS protein in the epithelial cells and in distant epithelial cells unexposed to the effective amount of UVA.

In some embodiments of the methods, the epithelial cells comprise or are tracheal epithelial cells and/or nasopharyngeal epithelial cells. Tracheal epithelial cells and/or nasopharyngeal epithelial cells can be exposed to, or are irradiated with, the UVA in some implementations of the methods.

In some embodiments of the methods, the epithelial cells comprise or are ciliated epithelial cells. Ciliated epithelial cells can be exposed to, or is irradiated with, the UVA in some implementations of the methods.

In some embodiments of the methods, the epithelial cells comprise or are ciliated tracheal epithelial cells and/or ciliated nasopharyngeal epithelial cells. Ciliated tracheal epithelial cells and/or ciliated nasopharyngeal epithelial cells can be exposed to, or are irradiated with, the UVA in some implementations of the methods.

In some embodiments of the methods, the epithelial cells comprise or are human nasal epithelial cells. In some embodiments of the methods, the epithelial cells comprise or are human trachea epithelial cells. Yet in some embodiments of the methods, the epithelial cells comprise or are human nasal epithelial cells and human trachea epithelial cells. Human nasal epithelial cells, human trachea epithelial cells, or both can be exposed to, or is irradiated with, the UVA in some implementations of the methods.

In some embodiments of the methods, the epithelial cells comprise or are human lung epithelial cells. Human lung epithelial cells can be exposed to, or is irradiated with, the UVA in some implementations of the methods.

In one embodiment, a method of increasing expression of mitochondrial antiviral signaling (MAVS) protein in epithelial cells in a subject includes exposing nasal epithelial cells, olfactory epithelial cells, oral epithelial cells, or combinations thereof to an effective amount of UVA for increasing the MAVS protein level in at least the exposed epithelial cells.

In another embodiment, a method of increasing expression of mitochondrial antiviral signaling (MAVS) protein in epithelial cells in a subject includes exposing nasal epithelial cells, olfactory epithelial cells, oral epithelial cells, or combinations thereof to an effective amount of UVA for increasing the MAVS protein level in the subject's trachea, bronchi, or both.

In yet another embodiment, a method of increasing expression of mitochondrial antiviral signaling (MAVS) protein in epithelial cells in a subject includes exposing nasal epithelial cells, olfactory epithelial cells, oral epithelial cells, or combinations thereof to an effective amount of UVA for increasing the MAVS protein level in epithelial cells in the subject's lung.

Yet in additional embodiments, the epithelial cells may also include or be one or more of urethral epithelial cells, bladder epithelial cells, vaginal epithelial cells, urogenital epithelial cells, rectal epithelial cells, gastrointestinal epithelial cells other than rectal epithelial cells, outer ear epithelial cells, and middle ear epithelial cells. Gastrointestinal system includes the organs of the mouth, pharynx (throat), esophagus, stomach, small intestine, large intestine, rectum, and anus. Therefore, gastrointestinal epithelial cells other than rectal epithelial cell may include one or more of oral mucosal epithelial cell, pharyngeal epithelial cells, esophageal epithelial cell, secretory epithelial cells that cover the surface of the stomach, and intestinal epithelial cells. Hence, in some embodiments, a method of increasing expression of mitochondrial antiviral signaling (MAVS) protein in epithelial cells in a subject includes exposing epithelial cells to an effective amount of UVA comprises exposing urethral epithelial cells, bladder epithelial cells, vaginal epithelial cells, urogenital epithelial cells, rectal epithelial cells, gastrointestinal epithelial cells other than rectal epithelial cells, outer ear epithelial cells, middle ear epithelial cells, or combinations thereof to an effective amount of UVA.

Preferably, exposing one area of epithelial cells to an amount of UVA, or a narrow band UVA, is effective for increasing MAVS protein level in not just this UVA-exposed area of epithelial cells, but also in distant areas of epithelial cells, including immediately adjacent, but unexposed, and farther distant areas of epithelial cells. More preferably, a continuum of epithelial cells (where cell-cell contact is involved) from the exposed area to the distant area, all exhibit the increased MAVS protein level.

One implementation provides that exposing (or irradiating) urethral epithelial cells increases MAVS protein expression in epithelial cells in the subject's bladder in a method disclosed herein.

Another implementation provides that exposing (or irradiating) vaginal epithelial cells increases MAVS protein expression in epithelial cells in the subject's uterus.

Another implementation provides that exposing (or irradiating) urogenital epithelial cells increases MAVS protein expression in epithelial cells in the subject's urethra or bladder.

Another implementation provides that exposing (or irradiating) rectal epithelial cells increases MAVS protein expression in epithelial cells in the subject's rectum or colon.

Another implementation provides that exposing (or irradiating) gastrointestinal epithelial cells other than rectal epithelial cells increases MAVS protein expression in epithelial cells in the subject's gastrointestinal tract.

Another implementation provides that exposing (or irradiating) outer ear epithelial cells increases MAVS protein expression in epithelial cells in the subject's middle or inner ear.

Yet another implementation provides that exposing (or irradiating) middle ear epithelial cells increases MAVS protein expression in epithelial cells in the subject's inner ear.

The subject in need of the methods, in some embodiments, are mammals including humans experiencing a microbial infection. In some embodiments, the subject in need of the methods are mammals including humans at risk of developing a microbial infection, or having been exposed to or having been in contact with another that has been infected or is suspected with the microbial infection, or having contacted an object detected with or suspected of having a presence of the microbe. Yet in some other embodiments, the subject in the methods exhibits one or more symptoms (or signs) of a microbial infection for no more than 10 days, or about 9, 8, 7, 6, 5, 4, 3, 2, or 1 day, or less than 24 hours. For example, the subject may exhibit one or more symptoms (or signs) of a microbial infection for no more than 7 days, and is selected to be subjected to a method disclosed herein. In another embodiment, the subject may exhibit one or more symptoms (or signs) of a microbial infection for no more than 5 days, and is selected to be subjected to a method disclosed herein. In another embodiment, the subject may exhibit one or more symptoms (signs) of a microbial infection for no more than 3 days, and is selected to be subjected to a method disclosed herein.

The microbial infection can be one or more of a viral infection, a bacterial infection, and a fungal infection, or caused by a parasite (e.g., Trichomonas vaginalis). Symptoms and signs of a microbial infection are known or accessible to one skilled in the medical art. Quite often, symptoms of a microbial infection may be associated with an inflammatory response.

Exemplary viral infections can be infected with, or caused by the presence of, coxsackievirus group B, coronavirus (e.g., coronavirus-229E), HIV, respiratory syncytial virus, parainfluenza viruses, respiratory adenoviruses, human herpesvirus (HHV), herpes simplex virus (HSV), human papillomavirus (HPV). Exemplary viral infections include but are not limited to common cold, influenza (flu), herpes, chickenpox, mumps, HPV infection, genital herpes, genital warts, measles, rubella.

Exemplary bacterial infections can be infected with, or caused by the presence of, Klebsiella pneumoniae, Escherichia coli, Clostridioides difficile, M. catarrhalis, Streptococcus pneumoniae, Haemophilus species, Streptococcus pyogenes, Staphylococcus aureus, Mycobacterium tuberculosis, Haemophilus influenza, group B Streptococcus, Staphylococcus, S saprophyticus, Proteus species, Enterococcus faecalis, Pneumococcus, or Salmonella. Exemplary bacterial infections include but are not limited to whooping cough, strep throat, sinusitis, bacterial rhinosinusitis, nasal vestibulitis, folliculitis, boils, pneumonia, tuberculosis, ear infection, otitis media, bacterial vaginosis, chlamydia, gonorrhea, urinary tract infection (UTI), cystitis.

Exemplary fungal infections can be infected with, or caused by the presence of, Candida (e.g., Candida albicans, Candida glabrata, Candida parapsilosis, and Candida tropicalis), Blastomyces, Cryptococcus gattii, Paracoccidioides, Coccidioides, Histoplasma, Aspergillus, or Cryptococcus neoformans. Exemplary yeast infections include but are not limited to athlete's foot, jock itch, ringworm, yeast infection (in one or more body parts such as vagina, mouth, throat, esophagus, ear, eye), candidiasis, thrush, onychomycosis, pneumocystic pneumonia, mucormycosis, and talaromycosis.

In some instances, the subject of a method disclosed herein is not infected with Klebsiella pneumoniae, Escherichia coli, Clostridioides difficile, Candida albicans, coxsackievirus group B, or coronavirus. In some instances, the subject of a method disclosed herein is infected with a microbe other than Klebsiella pneumoniae, Escherichia coli, Clostridioides difficile, Candida albicans, coxsackievirus group B, and coronavirus.

While the methods disclosed herein for a subject in need thereof may be accompanied by another medication, such as antibiotics, antiviral medications, antifungal medications, or a pain reliever to relief the pain with the symptoms of the infection, in some embodiments, the step of irradiating epithelial cells with UVA or exposing a body part of the subject to the UVA preferably does not require general anesthesia, regional anesthesia, local anesthesia, twilight anesthesia, or the administration of a sedative. In some embodiments, the methods disclosed herein do not include administering to the subject an anesthesia or a sedative prior to, during, and/or post-UVA exposure.

Different amounts or time period dosages of UV radiation may be administered depending on the type, severity, and location of the infection. For instance, in some embodiments, a higher intensity of UVA radiation may be administered for a shorter duration of time, or a lower intensity of UVA radiation may be administered for a longer duration of time, to realize a dosage with one type of epithelial cells, so as to result in a desired amount of increase in the MAVS protein level in the epithelial cells. As another instance, the light source may be manipulated to be placed at various distances from target epithelial cells based on the UVA intensity and/or being restricted by the space in the tissue, organ, or body parts to be irradiated.

The UVA light source intensity may be at least 1,000 microWatt/cm² (1,000 μW/cm², that is, 1 milliWatt/cm²), 1,100 microWatt/cm² (that is, 1.1 milliWatt/cm²), 2,000 microWatt/cm², 2,100 microWatt/cm², 2,200 microWatt/cm², 2,300 microWatt/cm², 2,400 microWatt/cm², 2,500 microWatt/cm², 2,600 microWatt/cm², 2,700 microWatt/cm², 2,800 microWatt/cm², 2,900 microWatt/cm², 3,100 microWatt/cm², 3100 microWatt/cm², 3,200 microWatt/cm², 1,000-5,000 microWatt/cm² or other suitable intensities depending on the application and other factors relevant to the treatment effectiveness. The inventors have confirmed that application of UV-A light is safe at intensities of at least about 5,000 microWatt/cm².

Various implementations of the methods disclosed herein do not cause UV-induced damage to the UV light exposed epithelial cells at least in the 335-350 nm. For example, an absence of UV-induced damage can be assessed by quantifying the live cell numbers or proliferation of the cells after a UV light exposure as compared to that before the exposure, or compare to control epithelial cells not exposed to the UV light exposure; and wherein a similar level of live cell number or cell proliferation to that of the control epithelial cells not exposed to the UV light indicates an absence of the UV-induced damage. As another example, an absence of UV-induced damage can be assessed by measuring 8-Oxo-2′-deoxyguanosine (8-OHdG) in cells, wherein a similar level of 8-OHdG in UV-exposed cells to that in cells not exposed to the UV light indicates an absence of UV-induced damage. In yet another example, an absence of UV-induced damage can be assessed in vivo (or in a mammalian subject exposed in an internal tissue to the UV light) by endoscopic evaluation for the absence of macroscopic evidence of mucosal erythema, friability, ulceration or bleeding, and/or by histological analysis of specimens for the absence of chronic/acute inflammation, cystitis, crypt abscesses, granulomata, ulceration, or dysplasia. Hence, in some embodiments of the methods disclosed herein further include a step for assessing or detecting an absence of UV-induced damage to the epithelial cells exposed to the UVA light and/or to the distant epithelial cells unexposed to the UVA light.

In some examples, the light will be delivered continuously. In other examples, the light will be incorporated into pulse therapy. In additional examples, the light will be delivered repeatedly, e.g., for two or more continuous exposures with a pause (no UVA exposure) in between exposures.

In some embodiments, the UVA light is administered, in each continuous exposure, for 1 minute, 2 minutes, 3 minutes, 4 minutes, 5 minutes, 6 minutes, 7 minutes, 8 minutes, 9 minutes, 10 minutes, 15, minutes, 18 minutes, 19 minutes, 20 minutes, 21 minutes, 22 minutes, 23, minutes, 24 minutes, 25 minutes, 26, minutes 27 minutes, 28 minutes, 29 minutes, 30 minutes, 60 minutes, 90 minutes, 120 minutes, or 160 minutes, any range of minutes between 1 and 160 minutes or other suitable times. In some embodiments, a threshold duration of UVA exposure is at least 20 minutes. In some embodiments, a threshold duration of UVA exposure is at least 15 minutes. In some embodiments, a threshold duration of UVA exposure is at least 10 minutes. In some embodiments, a threshold duration of UVA exposure is at least 5 minutes. In some embodiments, a threshold duration of UVA exposure is at least 3 minutes.

In addition, methods of the invention can include administering the UVA light for a threshold duration, or a total threshold duration when two or more exposures combined, of at least 1 minute, 2 minutes, 3 minutes, 4 minutes, 5 minutes, 6 minutes, 7 minutes, 8 minutes, 9 minutes, 10 minutes, 15, minutes, 18 minutes, 19 minutes, 20 minutes, 21 minutes, 22 minutes, 23, minutes, 24 minutes, 25 minutes, 26, minutes 27 minutes, 28 minutes, 29 minutes, 30 minutes, or 60 minutes.

Some embodiments provide that the methods include exposing epithelial cells for a first period of time, and subsequently exposing the epithelial cells for one or more additional periods of time, wherein each period of time is independently about 1-5 seconds, 5-10 seconds, 10-30 seconds, 30-60 seconds, 1-5 minutes, 5-10 minutes, 10-15 minutes, about 16 minutes, about 17 minutes, about 18 minutes, about 19 minutes, about 20 minutes, about 21 minutes, about 22 minutes, about 23 minutes, about 24 minutes, 25-30 minutes, 30-40 minutes, 40-50 minutes, 50-60 minutes, 60-90 minutes, 90-120 minutes, or 120-240 minutes; and wherein a period of time and its immediate prior or immediate following period of time may have a lapse period (where UVA is not administered), independently selected from seconds, minutes, hours, days, or other suitable lapse time.

In some embodiments, the UVA exposure, therapy or treatment is administered via a UVA light emitting diode (LED)-based catheter device which can be inserted into a body part (e.g., an endotracheal tube; the nasopharyngeal airway; the auditory pathway; the genital canal, etc.) to deliver UVA light. For example, a UVA LED-based catheter device may be controlled by a system to adjust and monitor the intensity and duration of wavelengths emitted from the UVA light sources.

Additional embodiments provide that an effective amount of the UVA therapy for epithelial cells is one, upon exposure to which the epithelial cells have at least 5%, 6%, 7%, 8%, 9%, 10%, 11%, 12%, 13%, 14%, 15%, 16%, 17%, 18%, 19%, 20%, 21%, 22%, 23%, 24%, or 25% increase in the MAVS protein level, compared to a reference value of the epithelial cells before the exposure to the UVA (“baseline”), or compared to a reference value of epithelial cells otherwise identical but not exposed to UVA. In other words, an effective amount of the UVA therapy for epithelial cells is one, upon exposure to which the MAVS protein expression level in the epithelial cells reaches (e.g., is measured to be at least) a target level. In some aspects, the target level is at least 5%, 6%, 7%, 8%, 9%, 10%, 11%, 12%, 13%, 14%, 15%, 16%, 17%, 18%, 19%, 20%, 21%, 22%, 23%, 24%, or 25% higher, compared to a reference value of the epithelial cells before the exposure to the UVA (“baseline”), or compared to a control. In some aspects, the target level of the MAVS protein expression is at least 20%, 21%, or 22% higher, compared to a reference value of the epithelial cells before the exposure to the UVA (“baseline”), or compared to a control.

The levels of MAVS protein in epithelial cells, e.g., the relative level of MAVS protein after UVA exposure compared to that before the UVA exposure, as well as the relative level of MAVS protein after a microbial infection but before a UVA exposure/therapy compared to that after the UVA exposure/therapy following the microbial infection, can, in some embodiments, be used as a guidepost to assess effectiveness of the UVA exposure and/or determine for the need of additional UVA exposure.

In some embodiments, a threshold increase of the MAVS protein level in epithelial cells (e.g., human ciliated tracheal epithelial cells) is at least 20%, 21%, or 22% after the UVA exposure compared to the baseline level (i.e., prior to the UVA exposure), or compared to otherwise identical epithelial cells that have not been exposed to UVA.

In some aspects, the epithelial cells subject to the UVA-induced threshold increase are not infected with a microbe, or are from an uninfected subject at least in the organ where the epithelial cells reside, wherein the threshold increase in the MAVS protein level may provide a prophylaxis effect.

In additional aspects, the UVA-induced threshold increase is an increase in the MAVS protein level wherein the epithelial cells are infected with a microbe. For example, the MAVS protein level in the epithelial cells after the UVA exposure/therapy following a microbial infection has a threshold increase compared to that in the epithelial cells following the microbial infection but before UVA therapy, or compared to that in the epithelial cells prior to the microbial infection and prior to the UVA therapy. This way, the threshold increase can reduce apoptosis in the epithelial cells infected with the microbe, and/or decrease proliferation or microbial load (e.g., viral load) in the epithelial cells.

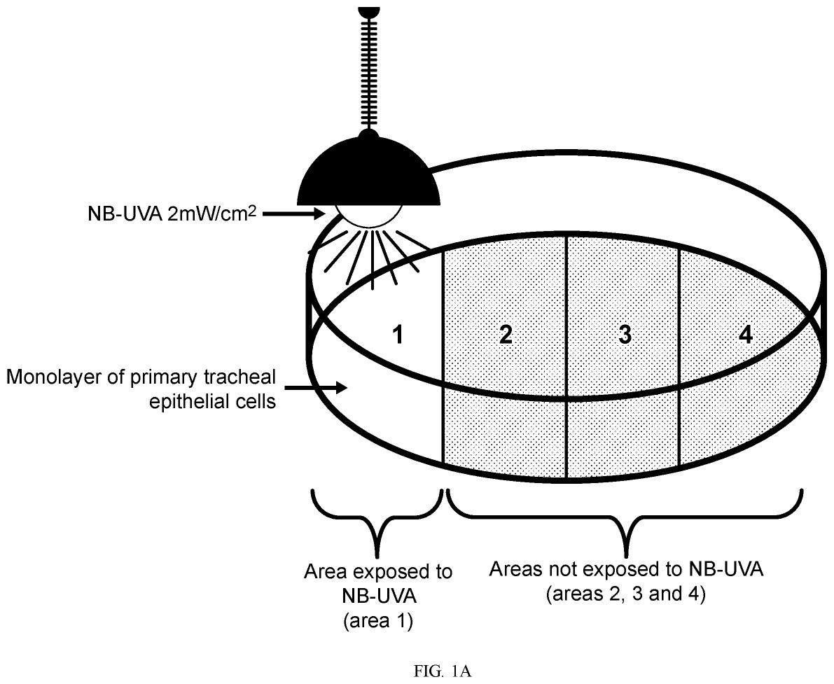

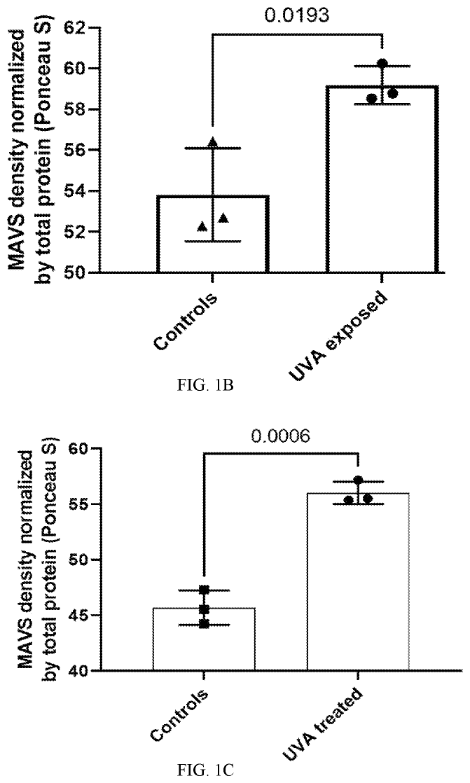

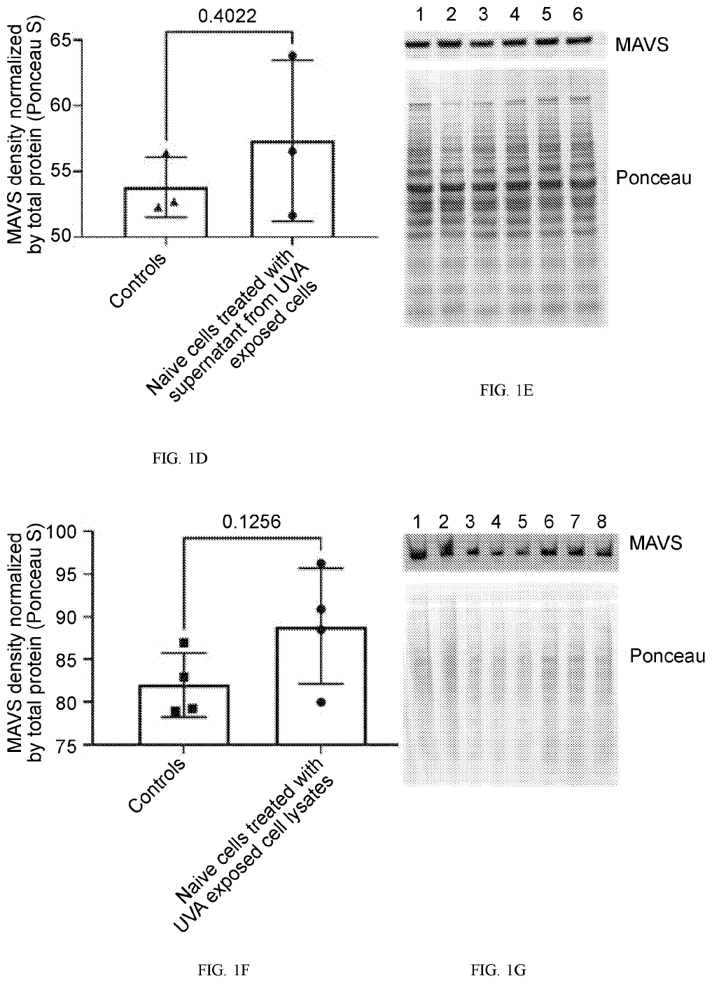

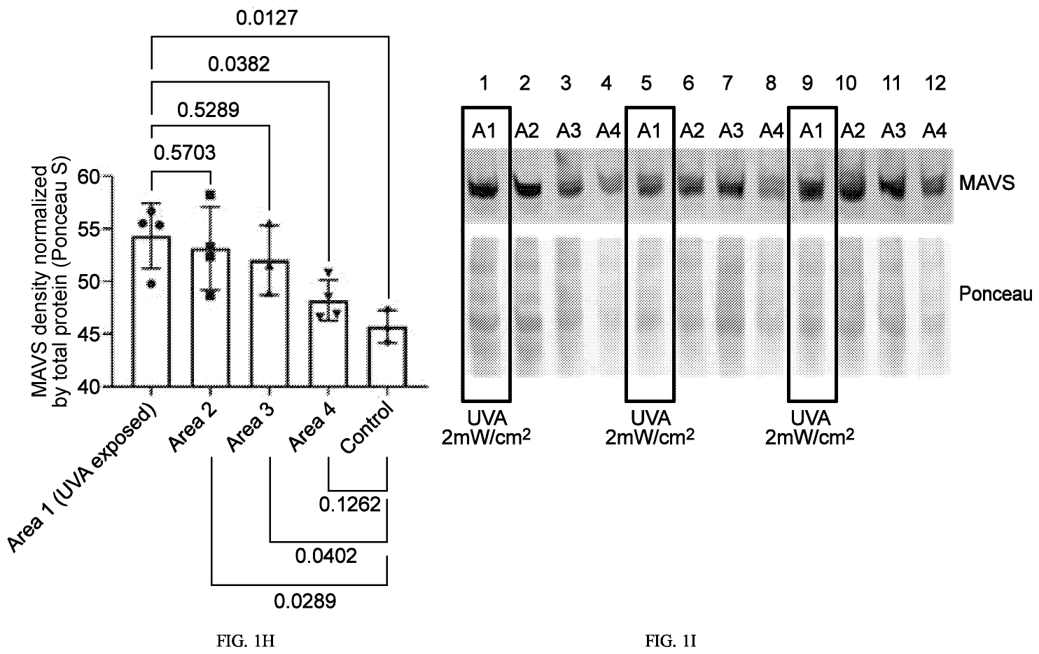

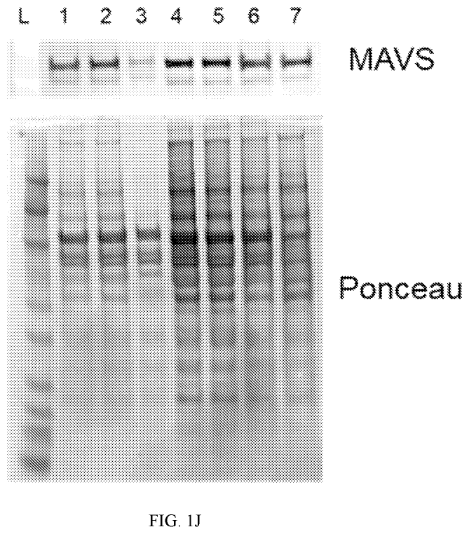

For instance, human tracheal epithelial cells exposed to, or irradiated with, a narrow-band UVA exposure of 20 minutes at an intensity of 2 mW/cm², in some embodiments, have an increase in the MAVS protein level of about 20%, 21%, 22%, or 23%, when the cells are at 100% confluency (as shown in FIG. 1C), mimicking healthy tracheal epithelium in mammals. In another instance, human tracheal epithelial cells exposed to, or irradiated with, a narrow-band UVA exposure of 20 minutes at an intensity of 2 mW/cm², in some embodiments, have an increase in the MAVS protein level of about 8%, 9%, or 10%, when the cells are at 30-40% confluency (as shown in FIG. 1B), mimicking damaged tracheal epithelium in mammals (possibly due to existing infection or other prior damage).

Therefore, in some instances, a prophylactic method may include irradiating mammalian tracheal epithelial cells, or the trachea or a mammal, with an amount of UVA therapy effective for increasing the MAVS protein level in the tracheal epithelial cells for at least 20%, 21%, 22%, or 23%, before the trachea is exposed to an infectious microbe. In some other instances, a prophylactic method may include irradiating mammalian tracheal epithelial cells, or the trachea or a mammal, with an amount of UVA therapy effective for increasing the MAVS protein level in the tracheal epithelial cells for at least 5%, 6%, 7%, 8%, 9%, or 10%, before the trachea is exposed to an infectious microbe. Optionally, the UVA intensity, duration, as well as peak wavelengths, may be adjusted for inducing a target level of the MAVS protein expression or a threshold increase in the MAVS protein expression.

In another instance, an intervention method may include irradiating mammalian tracheal epithelial cells, or the trachea or a mammal, with an amount of UVA therapy effective for increasing the MAVS protein level in the tracheal epithelial cells for at least 20%, 21%, 22%, or 23%, wherein the trachea has been exposed to an infectious microbe, so that the irradiation reduces apoptosis of the infected epithelium. In yet another instance, an intervention method may include irradiating mammalian tracheal epithelial cells, or the trachea or a mammal, with an amount of UVA therapy effective for increasing the MAVS protein level in the tracheal epithelial cells for at least 5%, 6%, 7%, 8%, 9%, or 10%, wherein the trachea has been exposed to an infectious microbe, so that the irradiation reduces apoptosis of the infected epithelium. Optionally, the UVA intensity, duration, as well as peak wavelengths, may be adjusted for inducing a target amount of MAVS protein increase.

In further aspects, when the MAVS protein level is below a target level or does not reach a threshold increase amount, the methods may include further exposing the epithelium or epithelial cells to the UVA, and optionally assaying the MAVS protein expression level from a sample of the epithelium or epithelial cells. In some implementations, when the assayed MAVS protein level continues to be below a target level or below a threshold increase amount, or when apoptosis of epithelial cells continues or microbial proliferation continues, the methods may include further exposing the epithelium or epithelial cells to the UVA or to a UVA of a higher intensity and/or longer duration than before; and optionally treating the subject with one or more antimicrobial medications. In other implementations, when the assayed MAVS protein level reaches a target level or reaches a threshold increase amount, and/or the apoptosis of epithelial cells decreases or ceases or the microbial proliferation decreases or ceases, the methods may discontinue the UVA exposure, or provide a similar UVA exposure as before for routine or prophylactic benefits.

In an additional embodiment, a method for administering UVA treatment in a subject in need thereof includes: assaying MAVS protein expression level in a biological sample obtained from a subject having been exposed to UVA treatment, and continuing to administer UVA treatment to the subject if the MA VS protein expression is below the subject's baseline level or compared to a control. In another embodiment, a method for administering UVA treatment in a subject in need thereof includes: assaying MAVS protein expression level in a biological sample obtained from a subject having been exposed to UVA treatment, and continuing to administer UVA treatment to the subject if the MAVS protein expression is below a target level. In some implementations, the subject is exposed to a first dose of UVA treatment before being assayed from the MAVS protein expression level in the subject's biological sample, and a second dose of UVA treatment is administered (as a “continued” administration) if the assayed MAVS protein expression is below the baseline, compared to a control, or compared to a target level; wherein the first dose and the second dose of UVA treatment may be the same, or different. For example, the second dose may be higher than the first dose, in intensity, duration, or both intensity and duration.

Hence, some embodiments provide the methods of assessing UVA treatment in a subject in need thereof, which includes assaying MAVS protein expression in a biological sample obtained from a subject having been exposed the UVA treatment, wherein the MAVS protein expression level higher than the subject's baseline level or higher than a control level indicates the treatment being effective. The biological sample, in various embodiments, includes epithelial cells or a portion of epithelium in respiratory cavity or canal (e.g., trachea, nasopharynx, oral cavity, bronchi), in the auditory pathway or sensory epithelium, in the genital tract or uterine luminal epithelium or bladder, in the eye, in the rectum/colon cavity, or another epithelium, from the subject.

Techniques of assaying MAVS protein expression level are available in the art, based on, including but not limited, anti-MAVS antibodies or conjugated anti-MAVS antibodies with a detectable label for one or more protein level quantifications, such as Western Blotting, ELISA, immunoprecipitation; isotope labeled MAVS protein or peptide, for mass spectrometry in quantification of the protein; or gene transcription level, e.g., mRNA quantifications. In some implementations, protein level quantifications are performed with biopsies obtained from the mammals.

The control in a method disclosed herein may be a reference value of the epithelial cells before exposure to the UVA, epithelial cells before contact with a pathogen, or population of epithelial cells not exposed to the amount of the UVA and not infected with a pathogen.

Various embodiments provide methods for increasing expression of mitochondrial antiviral signaling (MAVS) protein in epithelial cells, which include exposing epithelial cells to an effective amount of ultraviolet A (UVA), so as to increase expression of MAVS protein in the epithelial cells. In various aspects, the increase in the MAVS protein expression is compared to not having been exposed to the effective amount of the UVA or compared to a control.

In some embodiments, the epithelial cells comprise or consist tracheal epithelial cells. In some embodiments, the epithelial cells comprise or consist ciliated epithelial cells. In some embodiments, the epithelial cells comprise or consist ciliated epithelial cells. In some embodiments, the epithelial cells comprise or consist a combination of one or more of tracheal epithelial cells, ciliated epithelial cells, and ciliated epithelial cells. In some embodiments, the epithelial cells are human trachea epithelial cells. In some embodiments, the epithelial cells are human lung epithelial cells. In some embodiments, the epithelial cells are human nasal epithelial cells. In some embodiments, the epithelial cells are a combination of one or more of human trachea epithelial cells, human lung epithelial cells, and human nasal epithelial cells.

Various embodiments provide methods for increasing the expression of MAVS protein in a population of epithelial cells, comprising (1) exposing a first portion of the population of epithelial cells to an effective amount of UVA, so as to increase the expression in this first portion, and (2) contacting the first portion exposed to the UVA to a second portion of the population of epithelial cells not exposed to the UVA, so as to increase the expression in the second population.

Some embodiments provide methods for increasing the expression of MAVS protein in a population of epithelial cells, comprising (1) exposing a first portion of the population of epithelial cells to an effective amount of UVA, and (2) providing a second portion of the population of epithelial cells wherein the second portion has not been exposed to the UVA, or not exposing the second portion of the population of epithelial cells to UVA, wherein the first portion and the second portion are in cell-cell contact in the population, so that both the first portion and the second portion of the population of epithelial cells have an increased expression of MAVS protein compared to not having been exposed to the effective amount of the UVA or compared to a control.

Other embodiments provide methods for increasing the expression of MAVS protein in a population of epithelial cells, comprising exposing a first portion of the population of epithelial cells to an effective amount of UVA, obtaining cell lysates from the first portion of the population of epithelial cells following the exposure to the UVA, and exposing a second portion of the population of epithelial cells to the cell lysates obtained from the first portion of the population, wherein the second portion of the population has not been exposed to the UVA, so that both the first portion and the second portion of the population of epithelial cells have an increased expression of MAVS protein compared to not having been exposed to the effective amount of the UVA or compared to a control.

Further embodiments provide exposing a portion of trachea, lung (bronchi of lung), or nasal cavity of a subject to an effective amount of an UVA therapy by irradiating a portion of the trachea, the lung (bronchi of lung), or the nasal cavity to the UVA therapy, which results in an increased expression of MAVS protein in the ciliated epithelium of the exposed portion as well as an adjacent portion not exposed to the UVA therapy, thereby treating, reducing the severity, and/or reducing the risk of a respiratory microbial infection.

In some aspects, the second portion not previously exposed to the UVA has a surface area that is 10%-20%, 20%-30%, 30%-40%, 40%-50%, 50%-100%, 100%-200%, or 200%-300% of the size of the first portion exposed to the UVA. In some aspect, the second portion not previously exposed to the UVA has a quantity of cells that is 10%-20%, 20%-30%, 30%-40%, 40%-50%, 50%-100%, 100%-200%, or 200%-300% of the quantity in the first portion exposed to the UVA.

In some embodiments, an effective amount of an UVA therapy comprises an UVA intensity of 2-5 milliWatt/cm² (or mW/cm²), 5-10 mW/cm², 0.5-2 mW/cm², or 10-20 mW/cm². In various embodiments, an effective amount of an UVA therapy comprises two or more doses/exposures, each administered for a period of time, e.g., ranging from 30 seconds to 3 minutes, 3 minutes to 10 minutes, 10 minutes to 20 minutes, or 20 minutes to 30 minutes. In some embodiments, the UVA therapy is exposed to a target tissue at a distance between 0-1 cm, 0-1.5 cm, 0-2 cm, 0-2.5 cm, 0-3.0 cm, 0-3.5 cm, 0-4.0 cm, 0-5.0 cm, or 0-10 cm, or other similar and suitable ranges based on the intensity of the light and target pathogen. Hence, in some embodiments, exposing epithelial cells to an UVA light (e.g., as a therapy, a treatment, or an administration) so as to increase expression of MAVS protein in distant epithelial cells includes increasing expression of the MAVS protein in epithelial cells that are between 0-1 cm, 0-1.5 cm, 0-2 cm, 0-2.5 cm, 0-3.0 cm, 0-3.5 cm, 0-4.0 cm, 0-5.0 cm, 0-10 cm, or 0-30 cm, from the UVA light-exposed area/volume, or in some instances, from the periphery of the UVA light-exposed area/volume of epithelial cells.

In some aspects, the control is a reference value of the epithelial cells before exposure to the UVA. In some aspects, the control is a reference value from the epithelial cells before contact with a pathogen. In some aspects, the control is a reference value from a population of epithelial cells not exposed to the amount of the UVA and not infected with a pathogen.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1: Ultraviolet-A Light Increases Mitochondrial Anti-Viral Signaling Protein in Confluent Human Tracheal Cells Even at a Distance from the Light Source

Mitochondrial antiviral signaling (MAVS) protein mediates innate antiviral responses, including responses to certain coronaviruses such as severe acute respiratory syndrome coronavirus-2 (SARS-COV-2). We have previously shown that ultraviolet-A (UVA) therapy can prevent virus-induced cell death in human ciliated tracheal epithelial cells (HTEpC) infected with coronavirus-229E, and that UVA treatment results in an increase in intracellular levels of MAVS. In this study, we set out to determine the mechanisms by which UVA light can activate MAVS, and whether local UVA light application can activate MAVS at locations distant from the light source (such as via cell-to-cell communication). MAVS levels were compared in HTEpC exposed to 2 mW/cm² narrow band (NB)-UVA for 20 minutes and in unexposed controls, at 30-40% and at 100% confluency. MAVS levels were also compared in unexposed HTEpC treated with supernatants or lysates from UVA-exposed cells or from unexposed controls. Also, MAVS was assessed in different sections of confluent monolayer plates where only one section was exposed to NB-UVA. The results show that UVA increases the expression of MAVS protein. Cells in a confluent monolayer exposed to UVA were able to confer an elevation in MAVS in cells adjacent to the exposed section, and even cells in the most distant sections not exposed to UVA. In this study, human ciliated tracheal epithelial cells exposed to UVA demonstrate increased MAVS protein, and also appear to transmit this influence to distant confluent cells not exposed to light.

### Introduction

The human body has various defense mechanisms against infections, the most well-known of which involve innate immune responses where immune cells are recruited to sites of infection via cytokine signaling. Host intracellular responses to infection are also important, particularly in the defense against viruses. In the past decade, it has been discovered that mitochondria can mediate innate and adaptive immune responses via several mechanisms, including the production of mitochondrial anti-viral signaling (MAVS) protein.

The MAVS protein is primarily localized to the outer membrane of the mitochondria, and transduces signals from cytoplasmic retinoic acid-inducible gene I (RIG-I)-like receptors (RLRs) that recognize viral RNA. Specifically, after recognition and binding of viral components, the RLRs RIG-I and melanoma differentiation-associated gene 5 (MDA5) interact with MAVS, activating transcription factors that induce expression of proinflammatory factors and antiviral genes. However, some viruses have developed mechanisms to antagonize the activation of MAVS and evade this innate immune response. For example, the SARS-COV-2 transmembrane glycoprotein M is thought to antagonize MAVS, thus impairing MAVS-mediated innate antiviral responses.

We recently showed that application of UVA light, under specific conditions, to human ciliated tracheal epithelial cells infected with CoV-229E, significantly improved cell viability and prevented virus-induced cell death, and that this was accompanied by decreases in the levels of CoV-229E spike (S) protein. Moreover, cells treated with UVA light exhibited significantly increased levels of MAVS protein. This indicated that UVA may activate MAVS. Further, in a first-in-human clinical trial in ventilated subjects with coronavirus disease 2019 (COVID-19), a 20-minute endotracheal UVA treatment daily for 5 days resulted in significantly decreased respiratory SARS-COV-2 viral loads. Interestingly, despite time-limited localized UVA therapy in this trial, average log₁₀ changes in endotracheal viral load from baseline to day 6 was −3.2, indicating a potential antiviral phenomenon beyond immediate localized effects.

In this study, we explore the effects of narrow band (NB)-UVA light on MAVS expression in uninfected human ciliated tracheal epithelial cells in vitro. We also explore whether the effects of UVA light were limited to cells directly exposed to UVA, or were also seen in cells not directly exposed to UVA.

### Materials and Techniques

### NB-UVA Effects on MAVS

Primary human tracheal epithelial cells isolated from the surface epithelium of human trachea (HTEpC, lot no 454Z019.11, PromoCell GmbH, Heidelberg, Germany) were cultured at 37° C. (5% CO₂) in 60×15 mm standard tissue culture dishes (cat. 351007, Corning, NY, USA) with Airway Epithelial Cell Growth Medium (cat. C-21060, PromoCell) prepared with SupplementMix (cat. C-39165, PromoCell) and Gibco antibiotic-antimycotic solution (cat. 15240096, ThermoFisher Scientific, MA, USA).

Once the cells reached 10⁵ cells per plate (30-40% confluency), HTEpC were washed 3 times with sterile 1× PBS pH 7.4 (cat. 10010072, ThermoFisher), and fresh media was added to each plate. Cells were exposed to 2 mW/cm² of NB-UVA for 20 minutes based on previously validated ideal UVA irradiation levels. Unexposed cells were used as controls. After 24 hours the supernatants were collected, and cell were washed 3 times with sterile 1× PBS, pH 7.4. Following the removal of any remaining PBS, cells were lysed in the plate using 1 mL of RTL buffer from an AllPrep DNA/RNA/Protein isolation kit (Qiagen, Hilden, Germany). Experiments were performed in triplicate.

### NB-UVA Effects on MAVS Signal Transmission to Unexposed UVA-Naïve Cells

To determine whether the activation of MAVS caused by exposure to NB-UVA light could be transmitted to naïve, unexposed HTEPC, and to begin to elucidate the mechanisms involved, three experiments were performed:
To determine if an extracellular mediator was involved, supernatants from 30-40% confluent HTEpC that were exposed to NB-UVA were transferred to 30-40% confluent naïve HTEpC.
To determine if an intracellular mediator was involved, cell lysates from 30-40% confluent HTEpC that were exposed to NB-UVA (after supernatant removal) were transferred to 30-40% confluent naïve HTEpC.
To determine if cell-to-cell signaling was involved, areas of 100% confluent HTEpC exposed or not exposed to NB-UVA were analyzed.

### NB-UVA Effects on MAVS Signal Transmission Via Extracellular Mediators

Supernatants collected from UVA-exposed and control HTEpC from the previous experiment were transferred to a new 60×15 mm tissue culture dish containing 10⁵ naïve HTEpC (i.e. cells that were never exposed to UVA). Before receiving the supernatant from UVA-exposed or control cells, the naïve HTEpC were washed 3 times with sterile 1× PBS, pH 7.4. The PBS was completely removed, and 4 mL of the supernatant collected from UVA-exposed or control HTEpC were added to the naïve cells. After 24 hours of incubation, the cells were washed 3 times, and were then lysed in the plate using 1 mL of RTL buffer from an AllPrep DNA/RNA/Protein isolation kit (Qiagen). Experiments were performed in triplicate.

### NB-UVA Effects on MAVS Signal Transmission Via Intracellular Mediators

HTEpC were cultured at 37° C. (5% CO₂) in 60×15 mm standard tissue culture dishes (cat. 351007, Corning, NY, USA) with Airway Epithelial Cell Growth Medium (cat. C-21060, PromoCell) that included SupplementMix (cat. C-39165, PromoCell) and Gibco antibiotic-antimycotic solution (cat. 15240096, ThermoFisher Scientific, MA, USA).

Once the cells reached 10⁵ cells per plate (30-40% confluency), HTEpC were washed 3 times with sterile 1× PBS pH 7.4 (cat. 10010072, ThermoFisher), and fresh media was added to each plate. Cells were exposed to 2 mW/cm² of NB-UVA for 20 minutes. Unexposed cells were used as controls. After 24 hours, the cells were washed 3 times with sterile 1× PBS, pH 7.4, scraped from the culture dishes, and transferred to a 15 mL sterile tube. Cells were pelleted, and new fresh Airway Epithelial Cell Growth Medium was added. A single sterile 5 mm stainless steel bead (Qiagen) was added to each tube, and cells were lysed by vortexing the tube for 5 minutes. Lysates from UVA-exposed and control HTEpC were transferred to a new 60×15 mm tissue culture dish containing 10⁵ naïve HTEpC (i.e. HTEpC that had never been exposed to UVA). Before receiving the lysate from UVA-exposed or control cells, naïve HTEpC were washed 3 times with sterile 1× PBS, pH 7.4. The PBS was completely removed, and 4 mL of the lysate from either UVA-exposed or control HTEpC were added to the naïve cells. After 24 hours of incubation, the cells were washed 3 times with sterile 1× PBS and were then lysed in the plate using 1 mL of RTL buffer from an AllPrep DNA/RNA/Protein isolation kit (Qiagen). Experiments were performed four times.

### NB-UVA Effects on MAVS Signal Transmission Via Cell-to-Cell Signaling

HTEpC were cultured at 37° C. (5% CO₂) in 150 mm dishes (cat. 430599, Corning) with Airway Epithelial Cell Growth Medium (cat. C-21060, PromoCell) prepared with SupplementMix (cat. C-39165, PromoCell) and Gibco antibiotic-antimycotic solution (cat. 15240096, ThermoFisher) until they reached 100% confluence.

On the day of NB-UVA therapy, cells were washed twice with sterile 1× PBS, pH 7.4, and fresh media was added. Each 150 mm dish containing a 100% confluent monolayer of HTEpC was divided longitudinally into four sections, designated as areas 1, 2, 3 and 4, respectively (FIG. 1A). The NB-UVA emitting device was placed 2.3 cm from the bottom of the dish and approximately 2 mW/cm² of NB-UVA was applied to area 1 for 20 minutes. Experiments were performed four times.

To prevent UVA leakage to other parts of the plate during therapy, areas 2, 3 and 4 were covered with a sterile barrier which blocked the passage of light through the top and sides of the plate. During the course of the therapy NB-UVA intensity was constantly checked in unexposed areas (top, bottom, and sides) of the culture plates using a UV meter (SDL470, Extech, NH), to assure there was no UVA light in these areas. UVA-treated plates were then re-incubated at 37° C. (5% CO₂) for 24 h.

UVA-treated HTEpC plates were washed 3 times with sterile 1× PBS, pH 7.4, before harvesting the cells. 10 mL of sterile 1× PBS, pH 7.4, was added to the plate, and cells from area 4 were carefully scraped with a sterile Corning Cell Lifter (cat. 3008, Corning) and immediately transferred to a 15 mL sterile tube. Cells were pelleted at low speed (˜1000 RPM) and lysed with one mL RTL buffer from an AllPrep DNA/RNA/Protein isolation kit (Qiagen).

The remaining UVA-exposed HTEpC from areas 1, 2 and 3 (still attached to the plate) were washed 3 times with sterile 1× PBS, pH 7.4. 10 mL of sterile 1× PBS, pH 7.4, was added to the plate and cells from area 3 were carefully scraped and lysed as described above. The same process was used to harvest the cells from areas 2 and 1 (in this order).

### Protein Extraction and Western Blotting

AllPrep DNA/RNA/Protein Mini Kits (Qiagen) were used to extract total proteins from UVA-exposed and non-exposed HTEpC from all experiments, according to the manufacturer's protocol. Total proteins were quantitated using Qubit Protein Assays (ThermoFisher) and equal loads of total protein were separated on a NuPAGE 4-12% Bis-Tris mini gel (NP0336BOX, ThermoFisher) and then transferred onto a Biotrace NT nitrocellulose membrane (27376-991, VWR). Total proteins were stained with Ponceau S solution (P7170, Sigma-Aldrich). The membrane was blocked with tris-buffered saline containing 3% bovine serum albumin (cat. A7030, Sigma-Aldrich) and 0.1% Tween 20 (P1379, Sigma-Aldrich) (TBS-T), and incubated overnight at 4° C. with mouse anti-MAVS antibody (1:200; SC-166583, Santa Cruz Biotechnology) diluted in blocking solution. After washing in TBS-T, the membrane was then overlain with horseradish peroxidase (HRP)-conjugated goat anti-mouse IgG antibody (1:300; 5220-0286, SeraCare), washed in TBS-T, and exposed to enhanced chemiluminescence solution (RPN2235, GE Healthcare). Immunoreactive protein bands were imaged using an iBright FL1500 instrument (ThermoFisher) and analyzed using iBright Analysis software (ThermoFisher). Samples were normalized against total protein as determined from Ponceau S staining (MilliporeSigma, St. Louis, MO, US).

### Statistical Analysis

Graph construction and statistical analysis were performed with GraphPad Prism V. 9 (GraphPad Software, CA, USA). For all experiments, immunoreactive MAVS bands from nitrocellulose membranes were normalized against total protein (Ponceau S) before statistical analysis, using iBright Analysis software (ThermoFisher). MAVS relative densities (obtained after normalization) were compared between groups applying a non-paired t-test. Comparisons between each area from experiments with 100% confluent cell cultures were performed using paired t-test and ANOVA test. Significance level was set at p<0.05.

### Results

### Narrow Band-UVA (NB-UVA) Increases MAVS Protein Levels in Human Non-Confluent and Confluent Ciliated Tracheal Epithelial Cells

Levels of MAVS were analyzed in primary tracheal epithelial cells (HTEpC) at 30-40% confluency which were exposed to 2 mW/cm² NB-UVA for 20 minutes and in unexposed controls. Normalized MAVS levels, as detected by western blot, were increased in NB-UVA exposed cells when compared to unexposed controls (P=0.0193, FIG. 1B).

In addition, when primary tracheal epithelial cells were grown in 100% confluent monolayers (as opposed to 30-40% confluency), normalized MAVS levels in area 1 were also significantly increased following exposure to 2 mW/cm² NB-UVA for 20 minutes, when compared to levels in unexposed monolayers (P=0.0006, FIG. 1C, 1J).

MAVS is Activated by Cell-to-Cell Signaling after NB-UVA Exposure

When naïve 30-40% confluent HTEpC were treated with supernatants from NB-UVA exposed 30-40% confluent HTEpC, no changes in MAVS levels were observed (P=0.4022, FIG. 1D, 1E). However, when naïve 30-40% confluent HTEpC were incubated with cell lysates from NB-UVA exposed 30-40% confluent HTEpC, normalized levels of MAVS tended to increase (FIG. 1F, 1G, P=0.1256).

Next, levels of MAVS were analyzed in different areas of culture plates containing 100% confluent monolayers of HTEpC, after only one part of the plate (area 1) was exposed to 2 mW/cm² NB-UVA for 20 min (FIG. 1A). Normalized MAVS levels gradually increased from area 4 (farthest unexposed area) through area 1 (exposed to NB-UVA) (ANOVA P=0.08, FIG. 1H, 1I), and there was a statistically significant increase in MAVS levels in area 1 (exposed to NB-UVA) when compared to unexposed area 4 (P=0.0382, FIG. 1H, 1I). Importantly, levels of MAVS were also significantly increased in unexposed areas 2 and 3 when compared to controls from unexposed plates (P=0.0289 and P=0.0402 respectively, FIG. 1H). Normalized MAVS levels in area 4 (farthest unexposed area) also appeared to be higher than in controls, but did not reach statistical significance (P=0.1262, FIG. 1H).

In this study, we show that narrow band UVA light increases the expression of the MAVS protein in uninfected human ciliated tracheal epithelial cells in vitro. In addition, in a confluent monolayer culture of these cells, the induction of MAVS protein is transmitted to cells not directly exposed to NB-UVA light. This transmission does not appear to be due to a secreted extracellular mediator, but more likely results from direct cell-to-cell signaling, and possibly a cytosolic mediator.

External UVA therapy has long been used in the treatment of skin conditions such as psoriasis, eczema and skin lymphoma, for which it is FDA-approved. To explore the potential of internal UVA light therapy to treat microbial infections, we recently tested UVA efficacy against a variety of pathogens in vitro, and found that under controlled and monitored conditions, UVA light effectively reduced a variety of bacterial species (including Klebsiella pneumoniae, Escherichia coli, Clostridioides difficile, and others), the yeast Candida albicans, coxsackievirus group B, and coronavirus-229E. Importantly, we found that human ciliated tracheal epithelial cells that were infected with coronavirus-229E and then treated with NB-UVA light in vitro exhibited increases in MAVS protein and survived infection. These results indicated that the increased cell viability of coronavirus-229E-infected and UVA-treated cells, as compared to infected but untreated controls, might be due to activation of MAVS-mediated antiviral signaling pathways. In the present study, human ciliated tracheal epithelial cells were exposed to UVA light, without viral infection. The results confirmed that exposure to UVA light alone results in increased levels of the MAVS protein in these cells, demonstrating that this is a response to UVA light.

It is well recognized that the common cold, influenza and other viruses are seasonal and occur more often in winter and less in summer months. The mechanism for this is unclear, although data indicate that sunlight, and the production of vitamin D, may be important. Sunlight has historical importance in medicine—for example, during the H1N1 influenza pandemic of 1918-1919, it was indicated that the combination of access to sunlight and fresh air, together with strict hygiene and the use of face masks, may have lessened mortality among patients and staff at an ‘open-air’ hospital in Boston. A systematic review of data regarding vitamin D levels and the current COVID-19 pandemic indicates that sunlight and elevated vitamin D levels may improve outcomes. Although the trials selected for inclusion in the latter study had heterogeneous results, these and other historical data indicate that exposure to sunlight, and thus to UVA, may be beneficial in combating viral infections.

Under normal physiologic conditions, MAVS protein levels are low, due in part to binding of human antigen R as well as microRNAs to elements in the 3′UTR of the MAVS mRNA. Following recognition and binding of viral components, the N-terminal caspase recruitment domains (CARDs) of RIG-I-like receptors (RLRs) are ubiquitinated and bind to the CARD of MAVS, leading to aggregation of MAVS and activation of proinflammatory cytokines and antiviral interferon genes. However, viruses can also evade these pathways—for example, the membrane glycoprotein M of SARS-COV-2, the virus which causes COVID-19, can interact with MAVS and impair MAVS aggregation and activation of antiviral responses. In our preclinical studies, tracheal cells that were infected with CoV-229E and treated with UVA light also exhibited decreases in CoV-229E spike protein, which indicated to us that UVA light might also be an effective treatment for SARS-COV-2.

The primary site of SARS-COV-2 infection is the ciliated epithelial cells, associated with downstream characteristic bilateral ground-glass opacities. The acute respiratory viral infection and subsequent inflammatory responses can result in compromised pulmonary function and death. Secondary bacterial and fungal infections are also common, with ventilator-associated pneumonia (VAP) occurring in 31% of mechanically ventilated patients. To test the safety and efficacy of UVA light as a potential treatment for SARS-COV-2, we developed a new UVA light emitting diode (LED)-based catheter device which can be inserted into an endotracheal tube to deliver UVA light in critically ill COVID-19 subjects. In the first human study of mechanically ventilated COVID-19 subjects, all of whom had World Health Organization (WHO) symptom severity scores of 9 at baseline (10 is death), subjects who were treated with endotracheally-delivered UVA light (treated for 20 minutes daily for 5 days) exhibited an average log₁₀ decrease in SARS-COV-2 viral load of 3.2 (p<0.001) by day 6 of therapy in endotracheal aspirates, and these accelerated reductions in viral loads correlated with 30-day improvements in the WHO symptom severity scores. Moreover, the scale of the improvements, despite the fact that only small portions of the trachea were exposed to UVA light, indicated the possibility that the antiviral effects of UVA light might not be confined to cells directly exposed to UVA, but might also be transmitted to neighboring cells.

To explore the potential mechanisms underlying this transmission, we first took supernatants from UVA-exposed cells and added them to fresh plates of cells that were never exposed to UVA light. No increase in MAVS protein levels were seen in these cells, indicating that a secreted extracellular mediator was not involved. Next, to explore whether a cytosolic mediator was involved, we lysed UVA-exposed cells and non-exposed controls and added the lysates to fresh plates of cells that were never exposed to UVA light. There was a trend towards an increase in MAVS protein levels in naïve HTEpC incubated with lysates from UVA-exposed cells, but this did not reach significance. In contrast, when we compared MAVS levels in confluent monolayers of HTEpC directly exposed to UVA light and in adjacent areas from the same plate that were blocked from UVA light, we found that MAVS was not only increased in cells in area 1 (directly exposed to UVA light), but was also increased in cells in the adjacent areas 2, 3, and 4 which were blocked from direct UVA light, in a gradient that decreased with increasing distance from UVA-exposed cells. These findings confirm that an increase in MAVS in response to UVA light can be transmitted from directly exposed cells to neighboring unexposed cells, and indicate that cell-to-cell signaling is involved, although further work is required to determine the mechanisms involved.

Although SARS-COV-2 suppresses MAVS, UVA light exposure is shown here to override this suppression, and the mechanisms by which UVA light overrides this suppression, and potentially further exhibiting damage to single-stranded viral RNA, may be further explored. The effects of this MAVS activation might be important to study with in vivo models. Limited data indicate that MAVS and resultant intracellular production of interferon α might attract circulating immune cellular response to attack infected cells. Interestingly, in our previous in vitro study, CoV-229E caused precipitous cell death which was mitigated by UVA. This increased cell survival indicates that perhaps MAVS is a cell salvage pathway. This is also supported by the first in human study of UVA in intubated critically ill subjects with COVID-19. Two patients underwent bronchoscopy after 5 days of UVA application. There was no macroscopic evidence of inflammation. Further studies are needed to explore these concepts not addressed in this current study.

In conclusion, this study begins to unravel the possible mechanisms by which UVA light could influence innate intracellular immunity. The data herein shows that NB-UVA increases MAVS protein levels in human ciliated tracheal epithelial cells. This increase in MAVS protein appears to be transmissible to adjacent cells not directly exposed to UVA light. Further, our results indicate that MAVS signal transmission involves cell-to-cell communication, and possibly a cytosolic (but not a secreted extracellular) mediator. This finding may underlie the benefits of UVA seen in vitro and in human studies of critically ill patients with COVID-19. The findings could have wide-ranging implications for the treatment of SARS-COV-2, other coronaviruses and other RNA respiratory viruses such as influenza. Further work is needed to determine if this mechanism is an important factor in the seasonality of specific respiratory viral illnesses.

### Example 2. Study of NB-UVA Effects on Mitochondrial Antiviral Signaling (MAVS) Protein

Primary human tracheal epithelial cells isolated from the surface epithelium of human trachea (HTEpC, lot no 454Z019.11, PromoCell GmbH, Heidelberg, Germany) were cultured at 37° C. (5% CO₂) in 60×15 mm standard tissue culture dishes (cat. 351007, Corning, NY, USA) with Airway Epithelial Cell Growth Medium (cat. C-21060, PromoCell) prepared with SupplementMix (cat. C-39165, PromoCell) and Gibco antibiotic-antimycotic solution (cat. 15240096, ThermoFisher Scientific, MA, USA).

Once the cells reached 10⁶ cells per plate, HTEpC cultures were washed 3 times with sterile 1× PBS pH 7.4 (cat. 10010072, ThermoFisher), and fresh media was added to each plate. Cells were exposed to 2000 μW/cm² of NB-UVA for 0 (control) or 20 minutes every 24 hours for 1, 2, and 3 days. 24 hours after the last day of UVA therapy, the supernatants were collected, and cell cultures were washed 3 times with sterile 1× PBS, pH 7.4. After removing any remaining PBS, cells were lysed in the plate using 1 mL of RTL buffer from an AllPrep DNA/RNA/Protein isolation kit (Qiagen, Hilden, Germany).

### Protein Extraction and Western Blotting

AllPrep DNA/RNA/Protein Mini Kits (Qiagen) were used to extract total protein from UVA-exposed and unexposed tracheal cells, according to the manufacturer's protocol. Proteins were loaded onto a NuPAGE 4-12% Bis-Tris mini gel (NP0336BOX, ThermoFisher) and transferred onto a Biotrace NT nitrocellulose membrane (27376-991, VWR). Total proteins were stained with Ponceau S solution (P7170, Sigma-Aldrich). The membrane was blocked with tris-buffered saline containing 3% bovine serum albumin (cat. A7030, Sigma-Aldrich) and 0.1% Tween 20 (P1379, Sigma-Aldrich) (TBS-T) and incubated overnight at 4° C. with mouse anti-MAVS antibody (1:200; SC-166583, Santa Cruz Biotechnology) diluted in blocking solution. After washing in TBS-T, the membrane was then overlain with horseradish peroxidase (HRP)-conjugated goat anti-mouse IgG antibody (1:300; 5220-0286, SeraCare), washed in TBS-T, and exposed to enhanced chemiluminescence solution (RPN2235, GE Healthcare). Immunoreactive protein bands were imaged using an iBright FL1500 instrument (ThermoFisher).

### NB-UVA Effects on MAVS Via Secreted Molecules

Supernatants collected from UVA-exposed and control HTEpC cells were transferred to a new 60×15 mm tissue culture dish containing 10⁶ UVA unexposed HTEpC (naïve). Before receiving the supernatant from UVA-exposed or control cells, naïve HTEpC were washed 3 times with sterile 1× PBS, pH 7.4. The PBS was completely removed, and 4 mL of the supernatant collected from UVA-exposed or control HTEpC cells were added to the naïve cells. After 24 hours of incubation, the cells were washed 3 times, and proteins were extracted and analyzed as described previously.

### NB-UVA Effects on MAVS Via Cell-to-Cell Signaling

HTEpC were cultured at 37° C. (5% CO₂) in 150 mm dishes (cat. 430599, Corning) with Airway Epithelial Cell Growth Medium (cat. C-21060, PromoCell) prepared with SupplementMix (cat. C-39165, PromoCell) and Gibco antibiotic-antimycotic solution (cat. 15240096, ThermoFisher) until they reached 100% confluence.

On the day of NB-UVA therapy, cells were washed twice with sterile 1× PBS, pH 7.4, and fresh media was added. Each 150 mm dish containing a monolayer of HTEpC cells was divided longitudinally into four sections, designated as Areas 1, 2, 3 and 4, respectively (FIG. 1A). The NB-UVA emitting device was placed 2.3 cm of the bottom of the dish and approximately 2,000 μW/cm² of NB-UVA was applied to Area 1 for 20 minutes.

To avoid UVA leakage to other parts of the plate during therapy, areas 2, 3 and 4 were covered with a sterile device which blocked the passage of light through the top and sides of the plate (FIG. 1B). During the course of the therapy NB-UVA intensity was constantly checked in unexposed areas (top, bottom, and sides) of the culture plates using a UV meter (SDL470, Extech, NH), to assure there was no UVA light in these areas. UVA-treated plates were then re-incubated at 37° C. (5% CO₂) for 24 h.

UVA-treated HTEpC plates were washed 3 times with sterile 1× PBS, pH 7.4, before harvesting the cells. 10 mL of sterile 1× PBS, pH 7.4, was added to the plate, and cells from area 4 were carefully scraped with a sterile Corning Cell Lifter (cat. 3008, Corning) and immediately transferred to a 15 mL sterile tube. Cells were pelleted at low speed (˜1000 RPM) and lysed with one mL RTL buffer from an AllPrep DNA/RNA/Protein isolation kit (Qiagen).

The remaining UVA-exposed HTEpC cells from areas 1, 2 and 3 (still attached to the plate) were washed 3 times with sterile 1× PBS, pH 7.4. 10 mL of sterile 1× PBS, pH 7.4, was added to the plate and cells from area 3 were carefully scraped and lysed as described above. The same process was used to harvest the cells from areas 2 and 1 (in this order). Total proteins were extracted using an AllPrep DNA/RNA/Protein Mini Kit (Qiagen) and analyzed.

### Example 3. Narrow Band UVA Light (NB-UVA) Increases MAVS Levels in Human Ciliated Tracheal Epithelial Cells

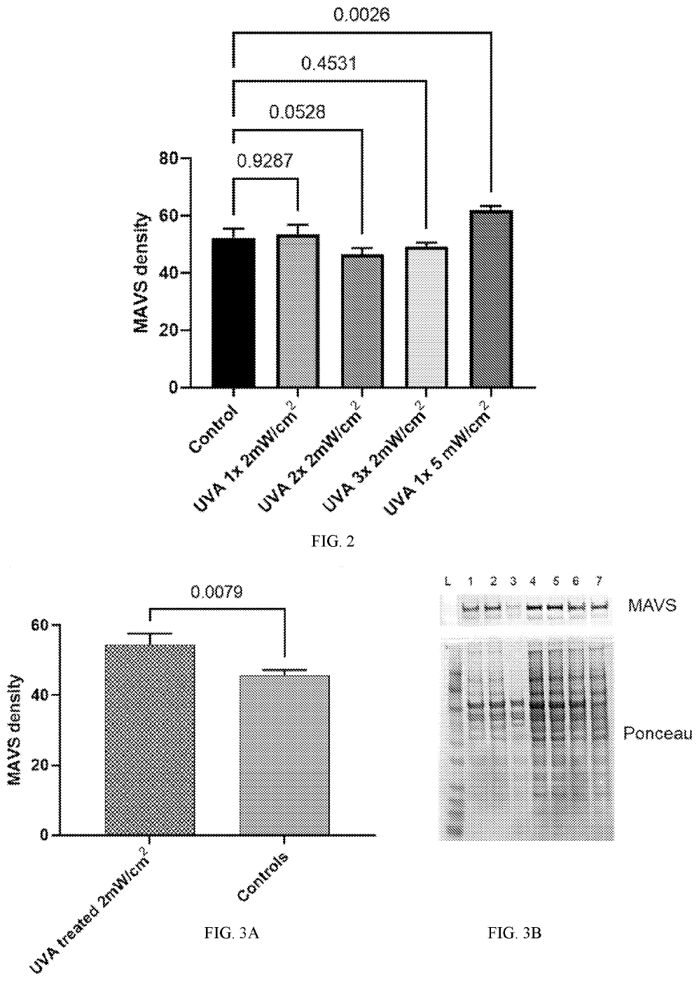

Levels of MAVS were analyzed in primary tracheal epithelial (HTEpC) cells at 30-40% confluency that were exposed to 2 mW/cm² NB-UVA (1 to 3 times) or to 5 mW/cm² (1 time), for 20 minutes per treatment. Normalized MAVS levels, as detected by western blot, increased when cells at 30-40% confluency were treated with 5 mW/cm² of NB-UVA light (P=0.0026), but no effects were observed when these cells were exposed to 2 mW/cm² of NB-UVA (P>0.05, FIG. 2).

In contrast, when primary tracheal epithelial cells were grown in 100% confluent monolayers (as opposed to 30-40% confluency), normalized MAVS levels were significantly increased following only one 20-min treatment with 2 mW/cm² NB-UVA, when compared to levels in unexposed monolayers (P=0.0079, FIGS. 3A and 3B).

### Example 4. MAVS is Activated by Cell-to-Cell Signaling after NB-UVA Exposure

In order to determine whether the activation of MAVS caused by exposure to NB-UVA light could be transmitted to naïve, unexposed HTEpC cells, and to begin to elucidate the mechanisms involved, three experiments were performed:
(1) To determine if a secreted factor was involved, supernatants from confluent NB-UVA exposed HTEpC cells were transferred to naïve HTEpC cells;
(2) To determine if a cytosolic factor was involved, cell lysates from exposed NB-UVA HTEPC cells (after supernatant removal) were transferred to naïve HTEpC cells; and
(3) To determine if cell-to-cell signaling was involved, areas of cells exposed to or not exposed to NB-UVA were analyzed from culture plates in which one part of the plate was exposed to NB-UVA (FIG. 1A).

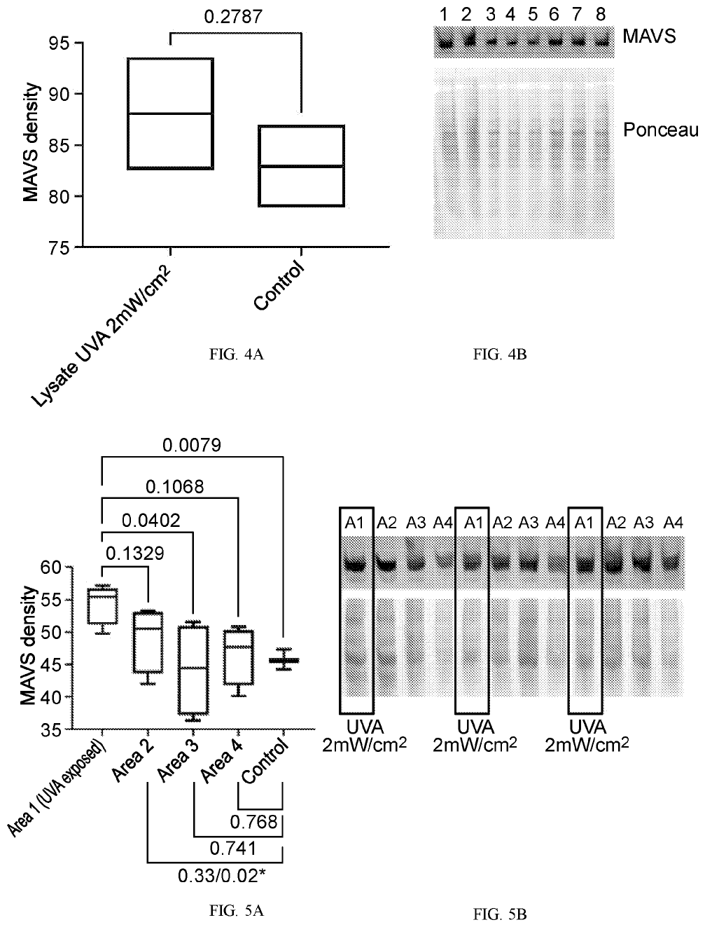

When supernatants from NB-UVA exposed HTEpC cells were transferred to naïve HTEpC cells, no changes in MAVS levels were observed. However, normalized levels of MAVS trended to increase in naïve HTEpC cells incubated with cell lysates prepared from NB-UVA exposed HTEpC cells (FIGS. 4A and 4B, P=0.2787).

Next, levels of MAVS were analyzed in different areas of culture plates containing 100% confluent monolayers of HTEpC cells, after only one part of the plate (Area 1) was exposed to 2 mW/cm² NB-UVA for 20 min (FIG. 1A). Normalized MAVS levels appeared to gradually increase from Area 4 (farthest unexposed area) through Area 1 (exposed to NB-UVA) (ANOVA P=0.07, FIGS. 5A and 5B). MAVS levels were increased in Area 1 (exposed to NB-UVA) when compared to unexposed Area 3 (P=0.04), and to a lesser extent when compared to unexposed Areas 2 and 4 (P=0.1329 and P=0.1068 respectively) (FIG. 4A). Importantly, levels of MAVS also appeared to be increased in unexposed Area 2 when compared to controls from unexposed plates (FIG. 5A), but the P-value only reached statistical significance when one outlier was omitted from the analysis (P=0.02).

In this study, the data indicates that UVA light increases the expression of the MAVS protein in human ciliated tracheal epithelial cells in vitro. In addition, in a confluent monolayer of these cells, the induction of MAVS is transmitted to cells not exposed to UVA light. This transmission does not appear to be due to a factor secreted into the media, but more likely results from direct cell-to-cell signaling, and possibly also a cytosolic factor.

External UVA therapy has long been used in the treatment of skin conditions such as psoriasis, eczema and skin lymphoma, for which it is FDA-approved. To explore the potential of internal UVA light therapy to treat microbial infections, we recently tested UVA efficacy against a variety of pathogens in vitro, and found that under controlled and monitored conditions, UVA light effectively reduced a variety of bacterial species (including Klebsiella pneumoniae, Escherichia coli, Clostridioides difficile, and others), the yeast Candida albicans, coxsackievirus group B, and coronavirus-229E. Importantly, we found that human ciliated tracheal epithelial cells that were infected with coronavirus-229E and then treated with UVA light in vitro exhibited increases in MAVS protein. These results indicated that the increased cell viability of coronavirus-229E-infected and UVA-treated cells, as compared to infected but untreated controls, might be due to activation of MAVS-mediated antiviral signaling pathways. In the present study, human ciliated tracheal epithelial cells were exposed to UVA light, without viral infection. The results confirmed that exposure to UVA light alone results in increased levels of the MAVS protein in these cells, confirming that this is a response to UVA light.

The common cold, influenza and other viruses appear seasonal. In particular, they occur more often in winter and less in summer months. The mechanism for this is unclear, although data indicate that sunlight, and the production of vitamin D, may be important. Sunlight has a historical importance in medicine—for example, during the H1N1 influenza pandemic of 1918-1919, it has been indicated that the combination of access to sunlight and fresh air, together with strict hygiene and the use of face masks, may have lessened mortality among patients and staff at an ‘open-air’ hospital in Boston. A systematic review of data regarding vitamin D levels and the current COVID-19 pandemic indicates that sunlight and elevated vitamin D levels may improve outcomes. Although the trials selected for inclusion in the latter study were mixed in their results, these and other historical data indicate that exposure to sunlight, and thus to UV, may be beneficial in combating viral infections.

Under normal physiologic conditions, MAVS protein levels are low, due in part to binding of human antigen R as well as microRNAs to elements in the 3′UTR of the MAVS mRNA. Following recognition and binding of viral components, the N-terminal caspase recruitment domains (CARDs) of RLRs are ubiquitinated and bind to the CARD of MAVS, leading to aggregation of MAVS and activation of proinflammatory cytokines and antiviral interferon genes. However, viruses can also evade these pathways—for example, the membrane glycoprotein M of SARS-COV-2, the virus which causes COVID-19, can interact with MAVS and impair MAVS aggregation and activation of antiviral responses. In our preclinical studies, tracheal cells that were infected with coronavirus-229E and treated with UVA light also exhibited decreases in coronavirus-229E spike protein, which indicated to us that UVA light might also be an effective treatment for SARS-COV-2.

The primary site of SARS-COV-2 infection is the lungs, which exhibit characteristic bilateral ground-glass opacities, and the acute respiratory viral infection and subsequent inflammatory responses can result in compromised pulmonary function and death. Secondary bacterial and fungal infections are also common, with ventilator-associated pneumonia (VAP) occurring in 31% of mechanically ventilated patients. To test the safety and efficacy of UVA light as a potential treatment for SARS-COV-2, we developed a new UVA LED based catheter device which can be inserted into an endotracheal tube to deliver UVA light in critically ill COVID-19 subjects. In a small-scale pilot study of mechanically ventilated COVID-19 subjects, all of whom had World Health Organization (WHO) symptom severity scores of 9 at baseline (10 is death), subjects who were treated with endotracheally-delivered UVA light (treated for 20 minutes daily for 5 days) exhibited an average log 10 decrease in SARS-COV-2 viral load of 3.2 (p<0.001) by day 6 of therapy in endotracheal aspirates, and these reductions in viral loads correlated with improvements in WHO symptom severity scores. Moreover, the scale of the improvements, despite the fact that only small portions of the trachea were exposed to UVA light, indicated the possibility that the effects of UVA light might not be confined to cells directly exposed to UVA, but might also be transmitted to neighboring cells. In this study, we found that when cells at 30-40% confluency were exposed to NB UVA light, no increase in MAVS was seen at lower UVA intensities (2 mW/cm²), and that higher UVA intensities (5 mW/cm²) were required before increases in MAVS were detected. In contrast, when cells were grown in a confluent monolayer, MAVS levels increased in response to lower intensities of UVA light (2 mW/cm²). This finding supported the hypothesis that the increase in MAVS in response to UVA light could be transmitted to other cells.

To explore the potential mechanisms underlying this transmission, we first took supernatants from UVA-exposed cells and added them to fresh plates of cells that were never exposed to UVA light. No increase in MAVS expression levels were seen in these cells, indicating that a secreted factor was not involved. Next, to explore whether a cytosolic factor was involved, we lysed UVA-exposed cells and non-exposed controls and added the lysates to fresh plates of cells that were never exposed to UVA light. There was a trend towards an increase in MAVS in naïve HTEpC cells incubated with lysates from UVA-exposed cells, but in this study this did not reach significance. When we compared MAVS levels in HTEpC cells directly exposed to UVA light and in adjacent areas from the same plate that were blocked from UVA light, we found that MAVS was not only increased in cells in Area 1 (directly exposed to UVA light), but was also increased in cells in the adjacent Areas 2, 3, and 4 that were blocked from direct UVA light, in a gradient that decreased with increasing distance from UVA-exposed cells. These findings confirm that an increase in MAVS in response to UVA light can be transmitted from directly exposed cells to neighboring unexposed cells, and indicate that cell-to-cell signaling is involved, although further work is required to determine the mechanisms involved.

UVA may have the potential to enhance innate cellular immunity to viruses. For example, SARS-COV-2 suppresses MAVS, and so understanding the mechanisms by which UVA light overrides this suppression would be important. This could include damage to single stranded viral RNA. In addition, the effects of this MAVS activation might be important to study with in vivo models. Limited data indicates that MAVS and resultant intracellular production of interferon γ might attract circulating immune cellular response to attack infected cells. Interestingly, in our previous in vitro study, coronavirus 229E caused precipitous cell death which was mitigated by UVA. This cell salvage indicates that perhaps MAVS is a cell salvage pathway (not cell lysis). This is also supported by the first in human study of UVA in intubated critically ill subjects with COVID-19. Two patients underwent bronchoscopy after 5 days of UVA application. There was no macroscopic evidence of inflammation of cellular denuding.

In conclusion, this study begins to unravel the possible mechanisms by which UVA light could influence innate cellular immunity. In this study, it appears that UVA increases the expression of MAVS in human ciliated tracheal epithelial cells. This expression appears transmissible to adjacent cells not exposed to light. Further, our results indicate that this transmission of an increase in MAVS involves cell-to-cell communication and possibly a cytosolic (but not a secreted) factor. This finding could support the benefits of UVA seen in vitro and in human studies of critically ill patients with COVID-19. The findings could have wide-ranging implications for the treatment of SARS-COV-2, other coronaviruses and other RNA respiratory viruses such as influenza. Further work is needed to determine if this mechanism is an important factor in the seasonality of specific respiratory viral illnesses.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method of increasing expression of mitochondrial antiviral signaling (MAVS) protein in epithelial cells in a subject in need thereof, comprising:
exposing epithelial cells to an effective amount of ultraviolet A (UVA), so as to increase expression of MAVS protein in the epithelial cells or in distant epithelial cells unexposed to the effective amount of UVA, wherein the increased expression of MAVS protein is compared to not having been exposed to the effective amount of the UVA or compared to a control.

2. The method of claim 1, wherein the epithelial cells comprise tracheal epithelial cells.

3. The method of claim 1, wherein the epithelial cells comprise ciliated epithelial cells.

4. The method of claim 1, wherein the epithelial cells comprise ciliated tracheal epithelial cells.

5. The method of claim 1, wherein the epithelial cells comprise human nasal epithelial cells, human trachea epithelial cells, or both.

6. The method of claim 1, wherein the epithelial cells comprise human lung epithelial cells.

7. The method of claim 1, wherein exposing epithelial cells to an effective amount of UVA comprises exposing nasal epithelial cells, oral epithelial cells, olfactory epithelial cells, or combinations thereof to the effective amount of UVA.

8. The method of claim 7, wherein exposing nasal epithelial cells, oral epithelial cells, olfactory epithelial cells, or combinations thereof increases MAVS protein expression in epithelial cells in the subject's trachea, bronchi, or both.

9. The method of claim 7, wherein exposing nasal epithelial cells, olfactory epithelial cells, oral epithelial cells, or combinations thereof increases MAVS protein expression in epithelial cells in the subject's lung.

10. The method of claim 1, wherein exposing epithelial cells to an effective amount of UVA comprises exposing urethral epithelial cells, bladder epithelial cells, vaginal epithelial cells, urogenital epithelial cells, rectal epithelial cells, gastrointestinal epithelial cells other than rectal epithelial cells, outer ear epithelial cells, middle ear epithelial cells, or combinations thereof to an effective amount of UVA.

11. The method of claim 10, wherein
exposing urethral epithelial cells increases MAVS protein expression in epithelial cells in the subject's bladder,
exposing vaginal epithelial cells increases MAVS protein expression in epithelial cells in the subject's uterus,
exposing urogenital epithelial cells increases MAVS protein expression in epithelial cells in the subject's urethra or bladder,
exposing rectal epithelial cells increases MAVS protein expression in epithelial cells in the subject's rectum or colon,
exposing gastrointestinal epithelial cells other than rectal epithelial cells increases MAVS protein expression in epithelial cells in the subject's gastrointestinal tract,
exposing outer ear epithelial cells increases MAVS protein expression in epithelial cells in the subject's middle or inner ear,
exposing middle ear epithelial cells increases MAVS protein expression in epithelial cells in the subject's inner ear, or
combinations thereof.

12. The method of claim 1, wherein the subject exhibits one or more symptoms of a microbial infection for no more than 3 days, 3-5 days, 5-7 days, or 7-10 days.

13. (canceled)

14. (canceled)

15. (canceled)

16. The method of claim 1, wherein the subject is not administered general anesthesia, regional anesthesia, local anesthesia, twilight anesthesia, or a sedative.

17. The method of claim 1, further comprising selecting the subject who exhibits one or more symptoms of a microbial infection as the subject in need thereof before exposing epithelial cells to an effective amount of UVA; wherein the microbial infection is a viral infection, bacterial infection, or fungal infection.

18. (canceled)

19. The method of claim 1, wherein the effective amount of the UVA comprises 5 milliWatt/cm² (mW/cm²) or more for a duration of 1 minute or more, or wherein the effective amount of the UVA comprises a UVA intensity of 5 mW/cm² or more.

20. The method of claim 1, wherein the effective amount of the UVA comprises 2-5 mW/cm² for a duration of at least 20 minute, or wherein the effective amount of the UVA comprises a UVA intensity of 2-5 mW/cm².

21. The method of claim 1, wherein the exposure to the effective amount of the UVA comprises exposing for a first period of time, followed by exposing for a subsequent period of time.

22. The method of claim 1, wherein the control is a reference value of the epithelial cells before exposure to the UVA, epithelial cells before contact with a pathogen, or population of epithelial cells not exposed to the amount of the UVA and not infected with a pathogen.

23. A method of assessing ultraviolet A (UVA) treatment in a subject in need thereof comprising:
assaying a biological sample obtained from a subject having been exposed to UVA treatment for mitochondrial antiviral signaling (MAVS) protein expression level,
wherein MAVS protein expression level higher than the subject's baseline level or higher than a control level indicates the treatment being effective.

24. A method of administering ultraviolet A (UVA) treatment in a subject in need thereof, comprising:
assaying mitochondrial antiviral signaling (MAVS) protein expression in a biological sample obtained from a subject having been exposed to UVA treatment; and
continuing to administer UVA treatment to the subject if MAVS protein expression is lower compared to the subject's baseline level, or compared to a control, or relative to a target level.

25. A method of administering ultraviolet A (UVA) treatment in a subject in need thereof, comprising:
performing the method of claim 1, in a subject having a low mitochondrial antiviral signaling (MAVS) protein expression as compared to a control, which is indicative of the subject needing the UVA treatment;
or
performing the method of claim 1, in a subject having a MAVS protein expression higher than the subject's baseline level, or compared to a control, which is indicative of the UVA treatment being effective.
